(19) United States
(12) Patent Application Publication
(10) Pub. No.: US 2013/0089570 A1
(43) Pub. Date: Apr. 11, 2013

(54) ORAL VACCINE COMPROMISING AN ANTIGEN AND A TOLL-LIKE RECEPTOR AGONIST

(71) Applicants: Nadia Ouaked, Quebec (CA); Martin Plante, Quebec (CA); Daniel Larocque, Quebec (CA); Corey Patrick Mallett, Quebec (CA)

(72) Inventors: Nadia Ouaked, Quebec (CA); Martin Plante, Quebec (CA); Daniel Larocque, Quebec (CA); Corey Patrick Mallett, Quebec (CA)

(73) Assignee: Glaxo Smith Kline Biologicals S.A., Rixensart (BE)

(21) Appl. No.: 13/701,237

(22) PCT Filed: Feb. 6, 2011
(86) PCT No.: PCT/EP2011/059167

§ 371 Date: Nov. 30, 2012

(30) Foreign Application Priority Data
Jun. 3, 2010 (GB) 1009273.2

(51) Int. Cl. A61K 39/39

(52) U.S. Cl. CPC A61K 39/39
USPC 424/209.1; 424/283.1; 424/234.1; 424/278.1

(57) ABSTRACT

The present invention provides an immunogenic composition comprising one or more antigens and a Toll-like receptor (TLR) agonist in an orally (e.g. sublingually) administered composition.

### FIELD OF THE INVENTION

The present invention provides immunogenic compositions suitable for oral delivery.

### BACKGROUND TO THE INVENTION

There is in general a need to increase patient compliance with vaccination as well as to improve ease of manufacture and transport of vaccines. Oral immunisation can address some of these needs and can be used to administer antigens in combination with adjuvants to induce antigen-specific immune responses, see for example WO99/21579.

### SUMMARY OF THE INVENTION

The present invention provides an immunogenic composition comprising one or more antigens and a Toll-like receptor (TLR) agonist in an orally administered composition and their use in medicine.

### BRIEF DESCRIPTION OF FIGURES

FIG. 1: A/Solomon Island virus-specific Ab responses induced in serum after s.I. administration of detergent split A/SI/3/2006 with or without TLR2 and/or TLR4 agonist as adjuvant. Mice were anesthetized and vaccinated s.I. with inactivated A/SI/3/2006 (7 or 14 μg)±SFOMP (5 μg), Pam3CysLip (10 μg) or CT (5 μg) as adjuvant at days 0 and 14. Two weeks after the second immunization, sera were collected and A/SI/3/2006 virus-specific Ab levels assessed by ELISA and the functionality of the serum IgG was evaluated by HI assay. Specific IgG concentrations are shown as ng/mL, and the number of mice per group having a protective HI titer (≧40) is indicated in the bar graph. NS=no significant differences in specific IgG levels vs. IgG levels in intramuscularly immunized mice. Each group had ten mice.

FIG. 2: A/Solomon Island virus-specific Ab responses induced in serum after s.I. administration of detergent split A/SI/3/2006 with or without TLR4±TLR2 agonist as adjuvant. Mice were anesthetized and vaccinated s.I. with inactivated A/SI/3/2006 (7 or 14 μg) adjuvanted with CRX527 (1 μg)±Pam3CysLip (5 μg) or CT (1 μg) at days 0 and 14. Two weeks after the second immunization, sera were collected and A/SI/3/2006 virus-specific Ab levels assessed by ELISA. The functionality of the serum IgG was evaluated by HI assay. Specific IgG levels are shown as geometric mean concentrations expressed as ng/ml, and 95% confidence limits are indicated. The number of mice per group having a protective HI titer (≧40) is indicated in the bar graph. NS=no significant differences in specific IgG levels vs. IgG levels in intramuscularly immunized mice. Each group had 5 to 10 mice.

FIG. 3: A/Solomon Island virus-specific Ab responses induced in serum after s.I. administration of detergent split A/SI/3/2006 adjuvanted with TLR agonists. Mice were anesthetized and vaccinated s.I. with inactivated A/SI/3/2006 (7.5 μg) adjuvanted with SFOMP (1 μg), Pam3CysLip (1 μg), CRX527 (1 μg), CRX642 (1 μg), MPL (1 μg), Flagellin (1 μg), CpG (1 μg) or CT (1 μg) at days 0 and 14. Two weeks after the second immunization, sera were collected and A/SI/3/2006 virus-specific Ab levels assessed by ELISA. The functionality of the serum IgG was evaluated by HI assay. Specific IgG levels are shown as geometric mean concentrations expressed as ng/ml, and 95% confidence limits are indicated. The number of mice per group having a protective HI titer (≧40) is indicated in the bar graph. NS=no significant differences in specific IgG levels vs. IgG levels in intramuscularly immunized mice (IIM). Each group had a total of 20 mice that were processed in 5 experiments of 4 animals/group. Due to technical difficulties, 2 pools of 4 mice each were excluded from the analysis of group immunized with CRX527.

### DETAILED DESCRIPTION

The present invention provides an immunogenic composition comprising one or more antigens and a Toll-like receptor (TLR) agonist in an orally administered composition.

The present invention provides an immunogenic composition comprising one or more antigens and a Toll-like receptor (TLR) agonist in an orally administered solid dispersing form designed to disintegrate rapidly in the oral cavity.

In a further embodiment of the invention, there is provided immunogenic composition as defined herein for use in a method of immunisation comprising the step of administering said composition orally, in particular sublingually. In a further embodiment of the invention there is provided an immunogenic composition as defined herein suitable for oral (in particular sublingual) administration comprising one or more antigens and a Toll-like receptor (TLR) agonist. In yet another embodiment of the invention, there is provided an orally (in particular sublingually) administered immunogenic composition as defined herein comprising one or more antigens and a Toll-like receptor (TLR) agonist.

In a further aspect of the invention, there is provided an immunogenic composition as defined herein for use in medicine.

In a further aspect of the invention, there is provided an immunogenic composition as defined herein for use in the treatment and/or prevention of disease.

The terms “oral administration”, “orally administered”, “oral vaccination”, “oral immunisation”, “oral delivery” as used herein are intended to refer to the application of antigens into the oral cavity wherein the immunogenic composition comprising an antigen is adsorbed in a manner which promotes an immune response at the mucosal tissue of the buccopharyngeal region. For the avoidance of doubt, these terms do not encompass administration of an antigen by ingestion i.e. wherein the antigen is swallowed or in any other way enters the stomach. In a particular embodiment, immunogenic compositions of the invention are administered sublingually, that is under the tongue.

An “orally (e.g. sublingually) administered composition” as used herein are intended to refer to a composition that is administered into the oral cavity wherein the immunogenic composition or at least antigenic components of the composition comprising an antigen are adsorbed in a manner which promotes an immune response at the mucosal tissue of the buccopharyngeal region. For the avoidance of doubt, these terms do not encompass compositions administered by ingestion i.e. wherein the antigen is swallowed or in any other way enters the stomach or any other means of administering an immunogenic composition known to the skilled person (for example intramuscular, intradermal, intranasal or transcutaneous administration). In a particular embodiment, immunogenic compositions of the invention are administered sublingually, that is under the tongue.

The orally administered immunogenic composition may be a liquid or a solid dose form. In a particular embodiment of the invention the immunogenic composition is in a solid dose form which disintegrates rapidly in the oral cavity. The immunogenic composition is in a solid dispersing form which disintegrates rapidly in the oral cavity. After disintegration, the components of the dosage form rapidly coat and are retained in contact with the mucosal tissues of the buccopharyngeal region, to include mucosal associated lymphoid tissue. This brings the antigenic components in contact with tissues capable of absorption of the antigen. In particular embodiment of the invention there is provided immunogenic composition is solid dose forms which disintegrate within about 1 to about 60 seconds, in particular about 1 to about 30 seconds, about 1 to about 10 seconds or about 2 to 8 seconds, of being placed in the oral cavity. Normally, the disintegration time will be less than 60 seconds which can be tested by following the disintegration method specified in United States Pharmacopoeia No. 23, 1995, in water at 37° C.

In a particular the orally administered immunogenic compositions comprise a mucoadhesive substance. Suitable solid dose forms are described in WO1999/021579 (EP1024824B1).

In a particular embodiment of the invention, there is provided a formulation comprising a mucoadhesive substance wherein the mucoadhesive substance is selected from the group: polyacrylic polymers, cellulose and derivatives thereof or natural polymers (e.g. gelatine, sodium alginate and pectin). In a particular embodiment the mucoadesive is selected from the group comprising chitosan or derivatives thereof, starch and derivatives thereof, hyaluronic and derivatives thereof, sodium alginate, gelatine, sodium polygalacturonate, dextran, mannan, cellulose film, synthetic non-degradable polymers, polyacrilic acid based polymers, carbopols or combinations thereof

In a further embodiment of the invention immunogenic compositions comprise in addition to the antigen(s) and adjuvant, matrix forming agents and secondary components. Matrix forming agents suitable for use in the present invention include materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes. Other matrix forming agents suitable for use in the present invention include sugars such as mannitol, dextrose, lactose, galactose and trehalose; cyclic sugars such as cyclodextrin; inorganic salts such as sodium phosphate, sodium chloride and aluminium silicates; and amino acids having from 2 to 12 carbon atoms such as a glycine, L-alanine, L-aspartic acid, L-glutamic acid, L-hydroxyproline, L-isoleucine, L-leucine and L-phenylalanine. One or more matrix forming agents may be incorporated into the solution or suspension prior to solidification. The matrix forming agent may be present in addition to a surfactant or to the exclusion of a surfactant. In addition to forming the matrix, the matrix forming agent may aid in maintaining the dispersion of any active ingredient within the solution or suspension. This is especially helpful in the case of antigens that are not sufficiently soluble in water and must, therefore, be suspended rather than dissolved.

In a further embodiment of the immunogenic compositions further comprise secondary components such as preservatives, antioxidants, surfactants, viscosity enhancers, colouring agents, flavouring agents, pH modifiers, sweeteners or taste-masking agents may also be incorporated into the composition. Suitable colouring agents include red, black and yellow iron oxides and FD & C dyes such as FD & C blue No. 2 and FD & C red No. 40 available from Ellis & Everard. Suitable flavouring agents include mint, raspberry, liquorice, orange, lemon, grapefruit, caramel, vanilla, cherry and grape flavours and combinations of these. Suitable pH modifiers include citric acid, tartaric acid, phosphoric acid, hydrochloric acid and maleic acid. Suitable sweeteners include aspartame, acesulfame K and thaumatic. Suitable taste-masking agents include sodium bicarbonate, ion-exchange resins, cyclodextrin inclusion compounds, adsorbates or microencapsulated actives.

The immunogenic compositions of the invention will comprise an antigen, which is capable capable of eliciting an immune response against a human or animal pathogen or a substance that causes pathogenesis in humans or animals.

The term ‘antigen’ is well known to the skilled person. An antigen can be a protein, polysaccharide, peptide, nucleic acid, protein-polysaccharide conjugates, molecule or hapten that is capable of raising an immune response in a human or animal. Antigens may be derived, homologous or synthesised to mimic molecules from viruses, bacteria, parasites, protozoan or fungus. The immunogenic compositions may include one or more antigens, in which embodiment the antigens may be taken from the same organism or from different organisms. In a particular embodiment of the invention the antigen is derived from influenza.

The immunogenic compositions of the invention comprise a Toll-like receptor agonist. By “TLR agonist” it is meant a component which is capable of causing a signalling response through a TLR signalling pathway, either as a direct ligand or indirectly through generation of endogenous or exogenous ligand (Sabroe et al, JI 2003 p 1630-5).

Toll-like receptors (TLRs) are type I transmembrane receptors, evolutionarily conserved between insects and humans. Ten TLRs have so far been established (TLRs 1-10) (Sabroe et al, JI 2003 p 1630-5). Members of the TLR family have similar extracellular and intracellular domains; their extracellular domains have been shown to have leucine-rich repeating sequences, and their intracellular domains are similar to the intracellular region of the interleukin-1 receptor (IL-1R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also posses the IL-1R domain in its cytoplasmic region, leading to NF-KB activation of cytokines; this Myd88 pathway is one way by which cytokine release is effected by TLR activation. The main expression of TLRs is in cell types such as antigen presenting cells (e.g. dendritic cells, macrophages etc).

Activation of dendritic cells by stimulation through the TLRs leads to maturation of dendritic cells, and production of inflammatory cytokines such as IL-12. Research carried out so far has found that TLRs recognise different types of agonists, although some agonists are common to several TLRs. TLR agonists are predominantly derived from bacteria or viruses, and include molecules such as flagellin or bacterial lipopolysaccharide (LPS).

In an embodiment the toll-like receptor agonist is a Toll like receptor (TLR) 4 agonist, preferably an agonist such as a lipid A derivative particularly monophosphoryl lipid A or more particularly 3 Deacylated monophoshoryl lipid A (3D-MPL).

3D-MPL is available under the trademark MPL® by GlaxoSmithKline Biologicals North America and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026).

OM 294 DP (3S, 9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462).

OM 197 MP-Ac DP (3S-, 9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127).

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants. In a particular embodiment of the invention the adjuvant is a TLR-4 agonist which is an AGP. In a particular embodiment, the TLR4 agonist is CRX524 or CRX527. CRX527 and CRX524 have been described previously (see U.S. Pat. No. 6,113,918; Examples 15 and 16, and WO 2006/012425 WO 2006/016997).

Other suitable TLR-4 ligands, capable of causing a signalling response through TLR-4 (Sabroe et al, JI 2003 p 1630-5) are, for example, lipopolysaccharide from gram-negative bacteria and its derivatives, or fragments thereof, in particular a non-toxic derivative of LPS (such as 3D-MPL). Other suitable TLR agonist are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, muramyl dipeptide (MDP) or F protein of respiratory syncitial virus. In one embodiment the TLR agonist is HSP 60, 70 or 90.

In a further embodiment of the invention the TLR agonist is a TLR2 agonist (Sabroe et al, JI 2003 p 1630-5). Suitably, the TLR agonist capable of causing a signalling response through TLR-2 is one or more of a lipoprotein, a peptidoglycan, a bacterial lipopeptide from M. tuberculosis, B. burgdorferi T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast. In a particular embodiment of the invention the TLR2 agonist In a particular embodiment of the invention the TLR2 agonist is the synthetic lipopeptide Pam3Cys-Lip (see for example Fisette et al., Journal of Biological Chemistry 278(47) 46252).

In a further embodiment of the invention, the immunogenic compositions of the invention comprise a TLR4 and a TLR2 agonist. In a particular embodiment, the immunogenic compositions of the invention comprise Shigella flexineri outer membrane protein preparations (SFOMP). In a particular embodiment, the immunogenic compositions comprise TLR4 agonist, such as an AGP (for example) CRX-527 and the TLR2 agonist Pam3CysLip.

Immunogenic compositions of the invention may comprise a TLR1, TLR2, TLR3, TLR4, TLR5, TLR6, TLR7, TLR8 or TLR9 agonist or a combination thereof.

In one embodiment of the present invention, a TLR agonist is used that is capable of causing a signalling response through TLR-1 (Sabroe et al, JI 2003 p 1630-5). Suitably, the TLR agonist capable of causing a signalling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam₃Cys) LP which mimics the acetylated amino terminus of a bacterial lipoprotein and OspA LP from Borrelia burgdorfei.

In an alternative embodiment, a TLR agonist is used that is capable of causing a signalling response through TLR-3 (Sabroe et al, JI 2003 p 1630-5). Suitably, the TLR agonist capable of causing a signalling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signalling response through TLR-5 (Sabroe et al, JI 2003 p 1630-5). Suitably, the TLR agonist capable of causing a signalling response through TLR-5 is bacterial flagellin or a variant thereof.

Said TLR-5 agonist may be flagellin or may be a fragment of flagellin which retains TLR-5 agonist activity. The flagellin can include a polypeptide selected from the group consisting of H. pylori, S. typhimurium, V. cholera, S. marcesens, S. flexneri, T. pallidum, L. pneumophilia, B. burgdorferei; C. difficile, R. meliloti, A. tumefaciens; R. lupine; B. clarridgeiae, P. mirabilis, B. subtilus, L. moncytogenes, P. aeruginosa and E. coli.

In a particular embodiment, the flagellin is selected from the group consisting of S. typhimurium flagellin B (Genbank Accession number AF045151), a fragment of S. typhimurium flagellin B, E. coli FliC. (Genbank Accession number AB028476); fragment of E. coli FliC; S. typhimurium flagellin FliC (ATCC14028) and a fragment of S. typhimurium flagellin FliC.

In a particular embodiment, said TLR-5 agonist is a truncated flagellin as described in WO2009/156405 i.e. one in which the hypervariable domain has been deleted. In one aspect of this embodiment, said TLR-5 agonist is selected from the group consisting of: FliC_{Δ174-400}; FliC_{Δ161-405} and FliC_{Δ138-405}.

In a further embodiment, said TLR-5 agonist is a flagellin as described in WO2009/128950

If the TLR-5 agonist is a fragment of a flagellin, it will be understood that said fragment will retain TLR5 agonist activity, and must therefore retain the portion of its sequence responsible for TLR-5 activation. It is known by the person skilled in the art that the NH₂ and COOH terminal domains of flagellin are important for TLR-5 interaction and activation, in particular for example amino acids 86-92 in Salmonella.

In an alternative embodiment, a TLR agonist is used that is capable of causing a signalling response through TLR-6 (Sabroe et al, JI 2003 p 1630-5). Suitably, the TLR agonist capable of causing a signalling response through TLR-6 is mycobacterial lipoprotein, di-acylated LP, and phenol-soluble modulin. Further TLR6 agonists are described in WO2003043572.

In an alternative embodiment, a TLR agonist is used that is capable of causing a signalling response through TLR-7 (Sabroe et al, JI 2003 p 1630-5). Suitably, the TLR agonist capable of causing a signalling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO02085905.

In an alternative embodiment, a TLR agonist is used that is capable of causing a signalling response through TLR-8 (Sabroe et al, JI 2003 p 1630-5). Suitably, the TLR agonist capable of causing a signalling response through TLR-8 is a single stranded RNA (ssRNA), an imidazoquinoline molecule with anti-viral activity, for example resiquimod (R848); resiquimod is also capable of recognition by TLR-7. Other TLR-8 agonists which may be used include those described in WO2004071459.

In one embodiment, there is provided an immunogenic composition of the invention wherein the TLR7/8 agonist an imidazoquinoline molecule, in particular an imidazoquinoline covalently linked to a phosphor- or phosphonolipid group. In a particular embodiment, immunogenic compositions of the invention comprise CRX642 (see WO2010/048520).

Immunostimulatory oligonucleotides or any other Toll-like receptor (TLR) 9 agonist may also be used. The preferred oligonucleotides for use in adjuvants or vaccines or immunogenic compositions of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

The CpG oligonucleotides utilised in the present invention may be synthesized by any method known in the art (for example see EP 468520). Conveniently, such oligonucleotides may be synthesized utilising an automated synthesizer.

Accordingly, in another embodiment, the adjuvant composition further comprises an additional immunostimulant which is selected from the group consisting of: a TLR-1 agonist, a TLR-2 agonist, TLR-3 agonist, a TLR-4 agonist, TLR-5 agonist, a TLR-6 agonist, TLR-7 agonist, a TLR-8 agonist, TLR-9 agonist, or a combination thereof.

In a particular embodiment of the invention, there is provided an immunogenic composition of the invention wherein the TLR agonist or at least one of the TLR agonists in a combination of TLR agonists is synthetic. By “synthetic” it is meant that the TLR agonist is not naturally occurring.

Immunogenic compositions of the invention may comprise a further immunostimulant, for example a saponin such as Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p 243-254). Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

The immunogenic compositions of the invention are suitable for use in medicine, accordingly, there is provided an immunogenic composition as described herein for use in medicine.

In a further embodiment, there is provided an immunogenic composition as described herein for use in a method of immunisation comprising the step of administering said composition orally (in particular sublingually), in particular to a human.

In a further embodiment, there is provided an immunogenic composition as described herein for use in the prevention and/or treatment of disease in particular in humans.

In a further embodiment, there is provided the use of an immunogenic composition as described herein in the manufacture of a medicament for the prevention and/or treatment of disease, in particular in humans.

Embodiments herein relating to “vaccine compositions” of the invention are also applicable to embodiments relating to “immunogenic compositions” of the invention, and vice versa.

The terms “comprising”, “comprise” and “comprises” herein are intended by the inventors to be optionally substitutable with the terms “consisting of”, “consist of” and “consists of”, respectively, in every instance.

### EXAMPLES

### Materials and Methods

### Animal Model and Vaccine Administration

Six to 8 week-old female BALB/c mice were obtained from Charles Rivers Canada. For sublingual immunization, mice were anesthetized by i.p. injection of ketamine and xylazine. Vaccines were administered by micropipette. The total volume of Ag plus adjuvant was kept to 8 μl to avoid swallowing effects. The i.m. injections were performed on thighs muscles in a volume of 50 μl. Mice were immunized on days 0 and 14 and sacrificed on day 28.

### Serum IgG ELISA

A final bleed was performed 2 weeks post last immunization (day 28). Serum was collected for specific IgG determination and the presence of functional serum antibodies. Determination of anti-A/Solomon/Island/3/2006 (A/SI/3/2006) IgG antibodies in mice was performed by ELISA using detergent split A/SI/3/2006 as coating antigen. Split Flu antigen was diluted at a final concentration of 0.5 μg/ml (25 ng/50 μl) in Coating Buffer (0.05M Carbonate/Bicarbonate, pH 9.6) and AffiniPure Goat Anti-Mouse IgG Fc-γfragment specific (Jackson Immuno Research) at a final concentration of 1.0 μg/mL (50 ng/50 μl) in Coating Buffer. Coating antigen and capture antibody were adsorbed during 4 hours at 20° C. onto Flat bottom 96-well polystyrene plates (Maxisorp, Nunc). Following the incubation, the plates were washed four times with DPBS (Dulbecco's phosphate buffered saline without Ca²⁺ or Mg²⁺; Gibco)/0.05% Tween 20 (Sigma). Plates were then incubated for 1 h at 20° C. with DPBS containing 1% bovine serum albumin (BSA, Sigma). Sera were diluted in buffer containing PBS, 0.05% Tween 20 and 1% BSA (sample dilution buffer), then added to split Flu-coated plates in serial dilutions and incubated for 16 h to 18 h at 4° C. Following the incubation, the plates were washed four times with PBS/0.05% Tween 20. The secondary antibody, a peroxidase-conjugated AffiniPure Goat Anti-Mouse IgG (Fc-γ fragment specific) diluted at 1/10000 in sample dilution buffer, was then added to each well and incubated for 30 min at 37° C. After a washing step (PBS/0.05% Tween 20), plates were incubated for 30 min at 20° C. with TMB peroxidase substrate (BD Biosciences). The reaction was stopped with 1M H₂SO₄ and read at 450 nm. Specific serum IgG concentration was calculated from a standard by SoftMaxPro by using a four-parameter equation and expressed as ng/ml.

### Mucosal Sample Preparation

Two weeks post second immunization, broncho-alveolar lavage (BAL), nasal wash, saliva, vaginal wash and feces were collected for antigen-specific IgA antibody determination. BAL and nasal wash samples were directly tested for IgA quantification. Saliva samples were extracted from the swab by adding 300 μL of the sample dilution buffer containing protease inhibitor cocktail (PIC) tablets complete mini (Roche) and samples were vortexed twice 15 seconds prior to being tested. Vaginal wash samples were diluted in 200 μL of sample dilution buffer containing PIC and bromelain (25 ug/mL) (Sigma), incubated 1 h at 37° C., and vortexed for 15 seconds before being assessed. Fecal pellets were kept on dry ice until the addition of PIC containing sample dilution buffer. Feces were weighted and resuspended in a volume in □L representing 5 times their weight in mg. Samples were homogenized (Kontes homogenizer) and centrifuged at 4° C. 7300 rpm during 5 min. Supernatant was collected and assessed by ELISA.

### IgA ELISA

Quantification of anti-A/SI/3/2006 IgG antibodies in mice was performed by ELISA similar to the one described for serum IgG determination. More specifically, coating was performed with split flu antigen diluted at a final concentration of 2 μg/ml (100 ng/50 μl) in Coating Buffer (0.05M Carbonate/Bicarbonate, pH 9.6) and Goat Anti-Mouse IgA (α-chain specific) (Sigma) at a final concentration of 1.0 μg/mL (50 ng/50 μl) in Coating Buffer. After overnight and blocking step, mucosal samples were added to split Flu-coated plates in serial dilutions and incubated for 16 h to 18 h at 4° C. Following the incubation, the secondary antibody, a peroxidase-conjugated AffiniPure Goat Anti-Mouse IgA (α-chain specific) diluted at 1/6000 in sample dilution buffer, was then added to each well and incubated for 30 min at 37° C. After incubation with TMB peroxidase substrate (BD Biosciences), the reaction was stopped with 1M H₂SO₄ and read at 450 nm. IgA concentration was calculated from a standard by SoftMaxPro by using a four-parameter equation and expressed as ng/ml.

### Hemaglutination Inhibition (HI) Assay

The HI assay was carried out on individual sera taken two weeks after the second immunization. Non-specific inhibitors were removed from serum by overnight treatment with receptor destroying enzyme (Sigma). Calcium saline solution was then added to achieve a 1:10 dilution, followed by incubation with 50% (v/v) solution of chicken or rooster pig red blood cells at 4° C. for 60 min to remove non-specific agglutinins. Treated serum was serially diluted in 25 μl of PBS and then incubated with an equal volume of PBS containing strain-specific influenza antigen (whole virus, containing 8 hemagglutinin units) for 45 min at room temperature. A 0.5% v/v suspension of red blood cells obtained from adult chicken or rooster were added and the mixture was incubated for another 45 min. Reactions were followed through visual inspection: a red dot formation indicates a positive reaction (inhibition) and a diffuse patch of cells a negative reaction (hemagglutination). As a negative control and in order to determine the background values of the assay serum samples of mice immunized with buffer were tested in parallel. All sera were run in duplicate. The HAI titer was recorded as the reciprocal of the last dilution that inhibited hemagglutination.

### Statistical Analysis

All statistical analyses were performed as followed. Values were transformed in log and analyzed for their Gaussian distribution with Shapiro-Wilk normality test. When the majority of group had a normal distribution or a value of skewness (−1≦1) and kurtosis (−1≦2) within acceptable limits, one-way ANOVA and Dunnett's Multiple Comparison test was performed. Otherwise, Kruskal-Wallis ANOVA and Dunn's Multiple Comparison test was done.

### Results and Discussion

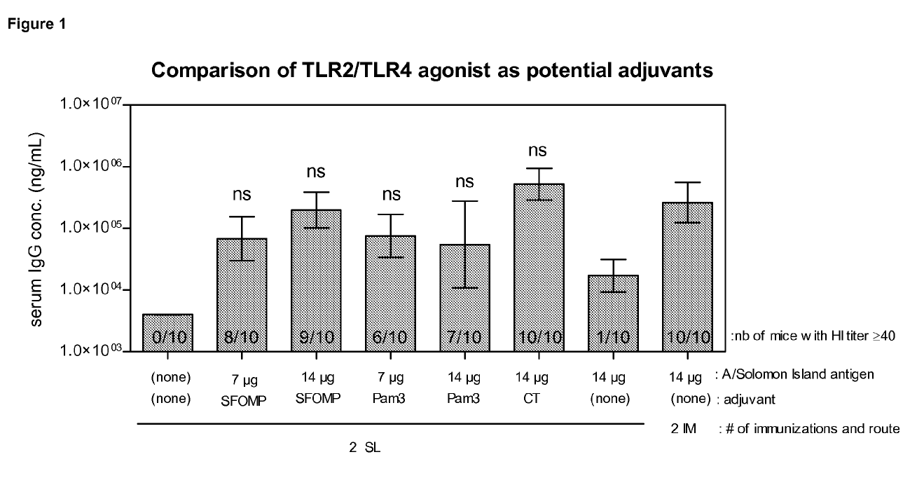

To determine the effectiveness of sublingual vaccination, new vaccine formulations using influenza antigens as model antigen adjuvanted with TLR2 and δ agonists were tested for their potency to elicit systemic and mucosal immune responses. A Shigella flexineri outer membrane protein preparations (SFOMP), a bacteria-derived TLR2/4 agonist and the synthetic lipopeptide Pam3CysLip, a TLR2 agonist, were first evaluated. BALB/c mice were immunized twice at 2-week intervals by the sublingual route with detergent-split A/SI/3/2006 virus adjuvanted with either SFOMP (5 μg), Pam3CysLip (10 μg), or with cholera toxin (CT). Two weeks after the final immunization, the levels of virus-specific antibodies were measured by ELISA and HI assays. A/Solomon Islands-specific serum IgG antibodies were detected in anesthetized animals that were immunized with split antigen adjuvanted with SFOMP or Pam3CysLip. All sublingually immunized mice with adjuvanted formulations showed statistically similar IgG levels to the intramuscularly vaccinated group (FIG. 1). Adjuvantation of 7 μg of antigen with Pam3CysLip or 14 μg of antigen with SFOMP significantly increased the IgG levels when compared to unadjuvanted vaccine. The functionality of the serum IgG was demonstrated by HI assay and as shown in FIG. 1, adjuvantation of sublingual vaccine led to a HI assay titers that are theoretically associated to minimum of 60% of protection. This data suggested a potential use of TLR2/4 agonists in sublingual immunization.

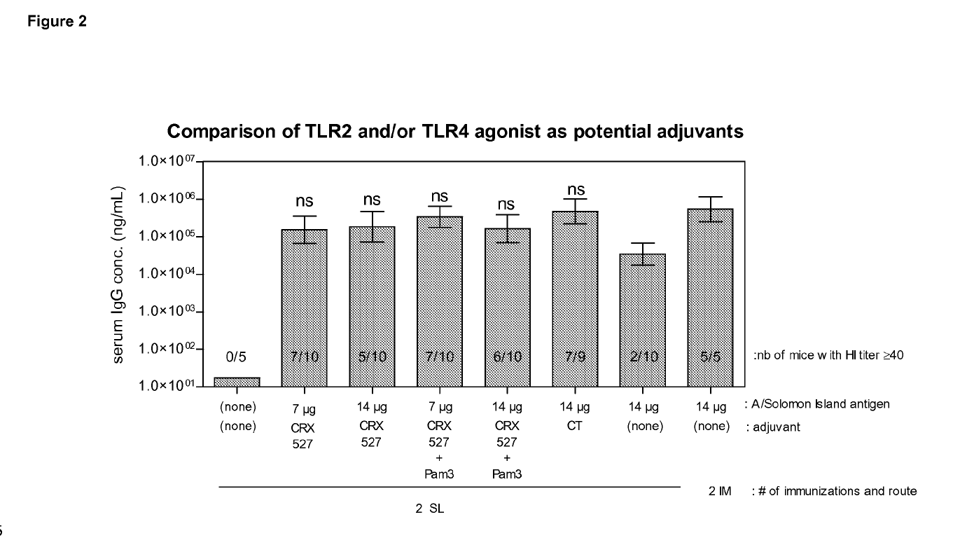

To confirm the potential of TLR2 and/or TLR4 agonists in sublingual vaccination, a pure synthetic TLR4 agonist (CRX527) was use alone or in combination with a pure TLR2 agonist (Pam3CysLip). CRX527 was investigated at 1 μg dose in a standard immunization regimen. Serum IgG ELISA analysis revealed that vaccine formulations comprising the TLR4 agonist CRX-527 (1 ug)±the TLR2 agonist Pam3CysLip (5 ug) and split influenza antigen are potent at eliciting antigen-specific serum IgG responses following sublingual immunization (FIG. 2). In this study, the adjuvantation effect of the sublingual vaccine was observed with each adjuvant. HI assay confirmed the presence of functional serum antibodies following sublingual administration of vaccine. Despite the high variability of the response within mice of the same group, there was a good association between the level of serum IgG and the HI titers.

In order to evaluate the mucosal antibody response in relevant compartments vs model antigen tested BAL, nasal wash and saliva were collected for antigen-specific IgA antibody determination. In addition, vaginal wash and feces were collected to investigate the extent of the mucosal immune response induced by the sublingual route. IgA ELISA analyses showed that A/SI/3/2006 vaccine formulations based on the TLR4 agonist±TLR2 agonist are potent at eliciting mucosal immune response when sublingually administered. As shown in Table 1, antigen-specific IgAs were detected in all mucosal compartments with the highest levels being in vaginal wash and fecal pellet samples. Any sublingually delivered vaccine, including unadjuvanted formulation, induced antigen specific response in the feces. Adjuvantation of the Solomon Islands detergent-split antigen offered at least a two-fold increase in the levels of antigen-specific IgAs.

TABLE 1
A/Solomon Island virus-specific mucosal Ab responses after s.l. administration of detergent split
A/SI/3/2006 with or without TLR4 ± TLR2 agonist as adjuvant. Mice were anesthetized and vaccinated s.l.
with inactivated A/SI/3/2006 (7 or 14 μg) adjuvanted with CRX527 (1 μg) ± Pam3CysLip (5 μg) or CT (1 μg)
at days 0 and 14. Two weeks after the second immunization, mucosal samples were collected and A/SI/3/2006
virus-specific IgA levels assessed by ELISA. Specific IgG levels are shown as geometric mean concentrations
expressed as ng/ml, and 95% confidence limits are indicated. Dunnett's Multiple Comparison Test was
performed.

| | | IgA concentration in mucosal samples | | | | |
|---|---|---|---|---|---|---|
| | | GeoMean (ng/mL) | | | | |
| | | Lower-Upper 95% Cl | | | | |
| | Dose | | | | | |
| Vaccine | of Ag | BAL ^{B} | Nasal Wash ^{B} | Saliva ^{B} | Vaginal Wash ^{B} | Feces ^{B} |
| PBS | None | 1.53 | 1.15 | 1.76 | 2.00 | 4.00 |
| | | 1.03-2.26 | 0.78-1.69 | 1.61-1.92 | 2.00-2.00 | 4.00-4.00 |
| CRX527 | 7 μg | 7.43*** | 11.88* | 7.53 | 81.01*** | 48.40*** |
| (1 μg) | | 4.04-13.65 | 5.47-25.79 | 2.83-20.04 | 29.54-222.10 | 18.93-123.80 |
| | 14 μg | 3.67 | 8.22 | 5.13 | 120.80*** | 75.71*** |
| | | 1.80-7.50 | 2.70-24.97 | 2.52-10.44 | 35.32-413.20 | 36.66-156.40 |
| CRX527 (1 μg) | 7 μg | 3.66 | 17.99** | 13.76* | 164.10*** | 100.10*** |
| + | | 1.47-9.10 | 6.76-47.86 | 5.77-32.85 | 97.54-275.90 | 52.02-192.70 |
| Pam3 CysLip | 14 μg | 9.12*** | 15.94** | 8.98 | 48.12*** | 95.90*** |
| (5 μg) | | 5.34-15.55 | 7.91-32.11 | 4.81-16.76 | 16.63-139.20 | 60.73-151.40 |
| CT (1 μg) | 14 μg | NA | 32.86** | 38.57*** | 101.90*** | 205.30*** |
| | | | 13.88-77.79 | 19.98-74.45 | 21.49-482.90 | 102.90-409.40 |
| Unadjuvanted | 14 μg | 1.12 | 3.93 | 2.73 | 10.71 | 22.82** |
| SL | | 0.67-1.88 | 1.17-13.25 | 1.67-4.46 | 4.42-25.91 | 10.56-49.33 |
| IM 2 doses | 14 μg | 0.87 | 2.00 | 2.00 | 2.00 | 4.00 |
| | | 0.87-0.87 | 2.00-2.00 | 2.00-2.00 | 2.00-2.00 | 4.00-4.00 |

Significant differences are indicated as followed: * = P≧0.05, * = P≧0.01 and * = P≧0.001 vs. intramuscularly immunized mice. Each group had 5 to 10 mice.
NA = Due to technical difficulties sample is not available.

To identify a potent antigen/adjuvant vaccine formulation, sublingual immunogenicity studies were performed with A/SI/3/2006 detergent split antigen adjuvanted with 7 candidate adjuvants candidates 1 μg dose). Intramuscular (IM) immunization was performed as a benchmark to determine the success of sublingual immunization. Since the marketed Flu vaccine is given as a one shot vaccine, intramuscular immunization was given once, either on the day of the first immunization, or on the day of the second immunization.

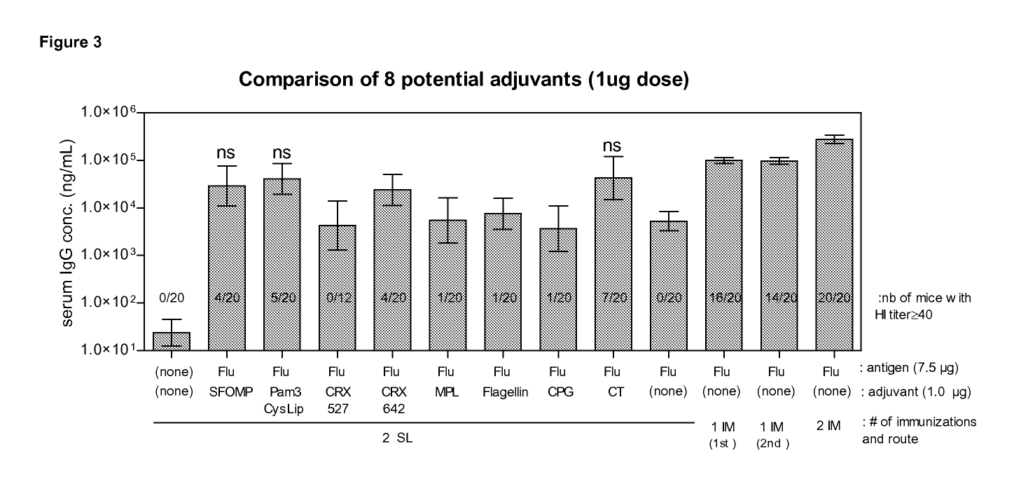

Serum IgG ELISA analysis revealed that 2 instillations of sublingually delivered unadjuvanted flu vaccine could elicit specific serum IgG response (GMC=5267 ng/mL) (FIG. 3). Adjuvantation of flu vaccine with SFOMP (GMC=28771 ng/mL), Pam3CysLip (GMC=40731 ng/mL), or CT (GMC=42343 ng/mL) induced similar specific IgG levels to intramuscular immunization given once either at day 0 or at day 14. In addition to adjuvantation with SFOMP or Pam3CysLip, which induces 5.5× and 7.7× increased IgG production in the serum compared to unadjuvanted sublingual flu vaccine, CRX642 (GMC=23966 ng/mL) also showed adjuvant effect and could induce significantly higher (4.6×) IgG level. Functional serum antibodies (HI titers≧40) could be induced following sublingual immunization. When animal were immunized twice with unadjuvanted vaccine, 1/40 animal showed a HI titer≧40. Increased number of mice having functional antibodies was observed in vaccine formulation adjuvanted with SFOMP (4/20), with Pam3CysLip (4/20), with CRX642 (4/20) or with CT (7/20). The discrepancy of these HI titers compared to the ones observed in the first sublingual study with SFOMP±Pam3CysLip with flu antigen is probably due to the route of immunization. As previously mentioned, a high coefficient of variation is always observed within the animals of the same group. To overcome this limitation is it planned to formulate the antigen with mucoadhesive compounds.

The mucosal immune response following sublingual immunization was investigated by IgA ELISA in several mucosal fluids. In contrast to IM immunization, sublingual immunization with adjuvanted split influenza antigen induces antigen-specific IgA in the BAL, nasal wash, saliva, vaginal wash and feces. Using Flu as a model antigen, the success criteria for sublingual immunization of mucosal antibody response in relevant compartments vs model antigen tested, would required IgA response in lung fluid, nasal wash and saliva.

BAL analyses revealed that low levels of specific IgAs are found in lung fluid following sublingual vaccination (Table 2). The highest IgA response was observed in animal immunized with CT adjuvanted flu vaccine (GMC=9.75 ng/mL). In addition to CT, Pam3CysLip (GMC=3.95 ng/mL), Flagellin (GMC=4.04 ng/mL) and CpG (GMC=4.10 ng/mL) adjuvanted vaccines induces significantly higher IgA BAL levels than IM immunization based on Kruskal Wallis and Dunn's multiple comparison test. Nasal wash analyses revealed that low levels of specific IgAs are found in lung fluid following sublingual vaccination. As in BALs, the highest IgA response was observed in animal immunized with CT adjuvanted flu vaccine (GMC=12.91 ng/mL). In addition to CT, only CpG (GMC=4.33 ng/mL) adjuvanted vaccines induces significantly higher IgA. Nasal Wash levels than IM immunization based on Kruskal Wallis and Dunn.s multiple comparison tests. CT was the only adjuvant tested inducing significantly higher levels of IgA in Nasal wash compared to unadjuvanted sublingual flu vaccine. Saliva analyses revealed that low levels of specific IgAs are found in saliva following sublingual vaccination. As in BAL and nasal wash, the highest IgA response was observed in animal immunized with CT adjuvanted flu vaccine (GMC=6.00 ng/mL). In addition to CT, Pam3CysLip (GMC=4.13/mL) adjuvanted vaccines induces significantly higher IgA saliva levels than IM immunization based on one way ANOVA and Dunnett's multiple comparison tests. Based on the success criteria for sublingual immunization of mucosal antibody response in relevant compartments vs model antigen tested, CpG, Pam3CysLip and Flagellin, represent potential candidates.

TABLE 2
A/Solomon Island virus-specific mucosal Ab responses after s.l. administration of detergent split
A/SI/3/2006 with or without TLR agonist as adjuvant. Mice were anesthetized and vaccinated s.l. with
inactivated A/SI/3/2006 (7.5 μg) adjuvanted with SFOMP (1 μg), Pam3CysLip (1 μg), CRX527 (1 μg),
CRX642 (1 μg), MPL(1 μg), Flagellin (1 μg), CpG(l μg) or CT (1 μg) at days 0 and 14. Two weeks after the
second immunization, mucosal samples were collected and A/SI/3/2006 virus-specific IgA levels assessed by
ELISA. Specific IgA levels are shown as geometric mean concentrations expressed as ng/ml, and 95%
confidence limits are indicated.

| | | IgA concentration in mucosal samples | | | | |
|---|---|---|---|---|---|---|
| | | GeoMean (ng/mL) | | | | |
| | | Lower-Upper 95% Cl | | | | |
| Vaccine | Dose | BAL ^{A} | Nasal Wash ^{A} | Saliva ^{B} | Vaginal Wash ^{B} | Feces ^{A} |
| PBS | None | 2.33 | 2.58 | 2.68 | 3.31 | 8.77 |
| | | 2.00-2.71 | 2.45-2.73 | 2.42-2.96 | 3.06-3.57 | 8.04-9.57 |
| SFOMP | 1 μg | 3.94 | 4.10 | 3.62 | 12.11*** | 16.05 |
| | | 2.86-5.42 | 3.16-5.32 | 3.00-4.35 | 6.31-23.22 | 10.20-5.26 |
| Pam3 | 1 μg | 3.95* | 5.25 | 4.13* | 16.21*** | 16.19 |
| CysLip | | 3.31-4.71 | 3.27-8.44 | 3.26-5.24 | 8.56-30.68 | 8.70-30.12 |
| CRX527 | 1 μg | 3.47 | 3.50 | 3.22 | 7.05 | 8.68 |
| | | 2.91-4.13 | 2.69-4.53 | 2.59-4.00 | 4.62-10.75 | 6.44-11.70 |
| CRX642 | 1 μg | 4.00 | 4.40 | 2.884 | 9.09* | 12.96 |
| | | 2.50-6.40 | 3.11-6.23 | 2.48-3.36 | 5.50-15.03 | 8.30-20.22 |
| MPL | 1 μg | 3.55 | 3.77 | 2.50 | 5.98 | 8.23 |
| | | 2.95-4.27 | 3.14-4.53 | 2.17-2.88 | 4.33-8.25 | 5.95-11.39 |
| Flagellin | 1 μg | 4.04* | 4.00 | 2.69 | 9.93** | 14.56 |
| | | 3.25-5.03 | 2.55-6.26 | 1.90-3.81 | 5.98-16.49 | 9.62-22.04 |
| CpG | 1 μg | 4.10* | 4.33** | 3.04 | 7.00 | 14.49 |
| | | 3.13-5.36 | 3.53-5.30 | 2.38-3.88 | 4.60-10.64 | 8.53-24.63 |
| CT | 1 μg | 9.75*** | 12.91*** | 6.00*** | 17.50*** | 49.67*** |
| | | 4.79-19.85 | 7.32-22.80 | 3.68-9.78 | 10.04-30.49 | 25.00-98.68 |
| Unadjuvant | None | 2.73 | 3.45 | 2.71 | 7.76** | 12.50 |
| ed SL | | 2.30-3.25 | 2.95-4.03 | 2.26-3.24 | 5.82-10.34 | 9.39-16.64 |
| IM 1 dose | None | 2.20 | 2.62 | 2.57 | 3.54 | 7.72 |
| | | 1.93-2.52 | 2.44-2.81 | 2.27-2.89 | 3.23-3.88 | 6.92-8.60 |
| IM 2 doses | None | 2.25 | 2.56 | 2.44 | 3.31 | 8.20 |
| | | 1.92-2.65 | 2.40-2.72 | 2.22-2.70 | 2.96-3.70 | 6.91-9.72 |

A: Dunn's Multiple Comparison Test was performed,
B: Dunnett's Multiple Comparison Test was performed.
Significant differences are indicated as followed * = P ≧ 0.05, * = P ≧ 0.01 and * = P ≧ 0.001 vs. intramuscularly immunized mice. Each group had 5 to 10 mice.

Vaginal wash analysis revealed that higher levels of specific IgAs can be detected in vaginal secretions following sublingual vaccination. As previously noted, IgA are undetectable following IM immunization and background level was set to a GMC=3.54 ng/mL. Sublingual unadjuvanted flu vaccine could induce 2.2 fold higher IgA levels (GMC=7.76 ng/mL) compared to IM immunization. Adjuvantation with SFOMP, Pam3CysLip, CRX642 or Flagellin highly increased the IgA response and therefore represent potential adjuvant candidates for antigens that require IgA in vaginal secretion. However, further studies are needed with the appropriate antigen. Specific IgAs could also be detected in feces following sublingual vaccination. Unadjuvanted sublingual vaccine induced similar fecal IgA levels to IM immunization. As indicated in table 2, only adjuvantation with CT significantly increased the IgA response compared to IM immunization.

### CONCLUSION

Several adjuvants have been tested for sublingual immunization of mice with Split Flu A/Solomon Island as a model antigen. Potential adjuvant candidates were shown to be SFOMP and Pam3CysLip. However, it is possible that the antigen concentration was still too high and CRX642 could, in lower antigen dose, represent a promising adjuvant. Based on functional assay, potential adjuvant candidates for sublingual immunization are SFOMP, Pam3CysLip and CRX642. Based on the success criteria for sublingual immunization of mucosal antibody response in relevant compartments vs model antigen tested, CpG, Pam3CysLip, Flagellin and CRX642 represent potential candidates. CMI analyses did not allow us to distinguish amongst the tested adjuvants in term of their Th1 cytokine production and cytokine pattern. All criteria combined, the most promising adjuvants for sublingual immunization are Pam3CysLip, CRX642 and Flagellin.

1. An immunogenic composition comprising one or more antigens and a Toll-like receptor (TLR) agonist in an orally administered composition.

2. An immunogenic composition according to claim 1 wherein the orally administered composition is a solid dispersing form designed to disintegrate rapidly in the oral cavity.

3. An immunogenic composition according to claim 1 wherein the adjuvant is selected from the group consisting of: TLR1 agonist, TLR2 agonist, TLR3 agonist, TLR4 agonist, TLR5 agonist, TLR7 agonist, TLR8 agonist, TLR9 agonist.

4. An immunogenic composition accordingly to claim 3 wherein the TLR agonist or at least one of the TLR agonists in a combination of TLR agonists is synthetic.

5. An immunogenic composition according to claim 3 wherein the adjuvant is a combination of a TLR4 and a TLR2 agonist.

6. An immunogenic composition according to claim 3 wherein the adjuvant is a TLR2 agonist.

7. An immunogenic composition according to claim 3 wherein the adjuvant is a TLR9 agonist.

8. An immunogenic composition according to claim 3 wherein the adjuvant is a TLR5 agonist.

9. An immunogenic composition according to claim 3 wherein the adjuvant is a TLR4 agonist.

10. An immunogenic composition according to claim 3 wherein the adjuvant is a TLR7/8 agonist.

11. An immunogenic composition according to claim 10 wherein the TLR7/8 agonist an imidazoquinoline molecule.

12. An immunogenic composition according to claim 10 wherein the TLR7/8 agonist is CRX642.

13. An immunogenic composition according to claim 1 comprising a further immunostimulant.

14. An immunogenic composition according to claim 2 wherein the solid dispersing form disintegrates within about 1 to about 60 seconds of being placed in the oral cavity.

15. An immunogenic composition according to claim 1 further comprising a mucoadhesive substance.

16. An immunogenic composition according to claim 15 wherein the mucoadhesive substance is selected from the group consisting of: polyacrylic polymers, cellulose derivatives or natural polymers.

17. An immunogenic composition according to claim 1 wherein the antigen is derived from influenza.

18. (canceled)

19. (canceled)

20. (canceled)

21. (canceled)

22. An immunogenic composition according to claim 5 wherein the adjuvant comprises a Shigella flexineri outer membrane protein preparation.

23. An immunogenic composition according to claim 5 wherein the adjuvant comprises an aminoalkyl glucosamine phosphate (AGP).

24. An immunogenic composition according to claim 5 wherein the adjuvant comprises Pam3cys.

25. An immunogenic composition according to claim 6 wherein the TLR2 agonist is Pam3cys.

26. An immunogenic composition according to claim 7 wherein the TLR9 agonist is an immunostimulatory oligonucleotide comprising one or more CpG motifs.

27. An immunogenic composition according to claim 8 wherein the TLR5 agonist is flagellin or a fragment thereof.

28. An immunogenic composition according to claim 3 wherein the TLR4 agonist is an AGP.

29. An immunogenic composition according to claim 10 wherein the imidazoquinoline molecule is covalently linked to a phosphor- or phosphonolipid group.

30. An immunogenic composition according to claim 13 wherein the further immunostimulant is Qs21.
